(19) United States
(12) Patent Application Publication
(10) Pub. No.: US 2009/0076417 A1
(43) Pub. Date: Mar. 19, 2009

(54) Glide Clip

(76) Inventor: Gregory Allen Jones, Dallas, TX (US)

Correspondence Address: CHALKER FLORES, LLP, 2711 LBJ FRWY, Suite 1036, DALLAS, TX, 75234, US

(21) Appl. No.: 12/188,879

(22) Filed: Aug. 8, 2008

Related U.S. Application Data
(60) Provisional application No. 60/954,755, filed on Aug. 8, 2007.

(51) Int. Cl. A61M 25/09

(52) U.S. Cl. 600/585; 604/164.13

(57) ABSTRACT

The present invention includes a self-tensioning apparatus for controlling the advancement and rotation of a surgical guideware comprising a torsion spring comprising one or more torsion spring handles; one or more arms connected to the torsion spring and one or more elastomeric guideware grippers, wherein compression of the one or more torsion spring handles reduced the compression between the one or more elastomeric grippers and the guideware; and an elongated, hollow housing comprising a first end and a second end, wherein the housing surrounds the spring tension metal clip comprising two or more opposing flexible pads positioned adjacent the spring tension handles that allow compression of the spring tension handles, the housing comprising a guideware guiding groove that extends from the first to the second end of the housing, wherein the one or more elastomeric guideware-grippers are aligned with and are positioned about the groove, wherein the one or more elastomeric grippers contact and grip the guideware in the groove when the one or more spring tension handles are not engaged, but release variably as the user compresses the flexible pads on the housing.

### CROSS-REFERENCE TO RELATED APPLICATIONS

This application claims priority to U.S. Provisional Application Ser. No. 60/954,755, filed Aug. 8, 2008, the entire contents of which are incorporated herein by reference.

### TECHNICAL FIELD OF THE INVENTION

The present invention relates in general to the field of devices that aid the surgeon during surgery, and more particularly, to devices, methods and kits for a guide clip used to control wires and catheters during surgical procedures.

### BACKGROUND OF THE INVENTION

Without limiting the scope of the invention, its background is described in connection with minimally invasive surgery.

Minimally invasive endovascular and interventional procedures of the cardiovascular, biliary, renal and gastrointestinal systems frequently require manipulation of a catheter, which is directed over a guide wire. The guide wire is often manipulated for directional positioning of the catheter by torqueing or rotating the guide wire. Many of the guide wires used for directional access are hydrophilic or lubricated by water for easier positioning through very tortuous or narrow vessels or bile ducts/ureters etc. These directional guide wires are rotated or torqued by a “torque device”.

These existing devices attempted in the past have required the physician to reach to the rear of the guide wire to back load the device over the wire demanding he use both hands and let go of the catheter to place or move the device. U.S. Pat. Nos. 5,137,517 and 5,312,338 allow placement at any point along the guide wire but require two hands to manipulate requiring the operator to turn his or her attention away from the operation in order to place or move the device. The operator is also forced to release the catheter in order to perform these maneuvers of the torque device, possibly losing position already gained with the guide wire.

Many other attempts have been made in designing a torque device to overcome back loading onto the guide wire but they have failed to maintain a cylindrical shape that is not cumbersome to rotate or apply and release with a single hand. Many of these devices have buttons or clasps that protrude from the device interfering with easy rotation. For example, U.S. Pat. No. 6,030,349 allows one handed placement onto the guide wire at any point but requires the operator to depress a protruding button to apply the device. The rotation of the device is then encumbered by the protruding button, which can also pinch surgical latex gloves, which then become entangled limiting ease of rotation. It also is dependent on gripping the guide wire in a channel instead of having jaws that directly grip the guide wire thereby allowing slippage limiting effective torque.

The devices attempted in the past have not allowed the operator to easily move the device along the guide wire with one hand, releasing and reattaching as the guide wire is advanced or withdrawn. U.S. Pat. No. 5,625,868 allows single handed movement along the guide wire but requires the operator to stop working and go to the rear of the guide wire to back load the device.

The devices attempted in the past have not offered an internal mechanism for wetting and wiping a hydrophilic guide wire automatically during use, requiring the operator to frequently stop and wet the wire with gauze or telfa material to maintain lubricity and clear blood clot and debris from the wire. Also, existing devices are not currently available in different sizes to better fit different guide wires or operator preferences. Therefore, there are no devices available currently that allow direct one-handed attachment onto any point along the guide wire, easy movement back and forth with one hand, cylindrical shape ideal for rotation, the ability to wet and wipe the guide wire and is available in different sizes to better fit different guide wires and operator preferences.

### SUMMARY OF THE INVENTION

The present invention is a torque device composed of two parts, a spring tensioned metal clip with rubber wire grippers housed in an outer casing with two opposing flexible areas allowing compression of the inner spring thereby releasing the tension and allowing attachment to a medical endovascular guide wire. The outer housing is filled with an absorbent foam material that when wet will both wet and wipe a hydrophilic guide wire. The present invention will allow single hand attachment to interventional radiology/cardiology (endovascular) guide wires for the purpose of torqueing (rotating) and advancing the guide wire. The present invention can be placed on and moved with one hand rather than all current devices two hands design. The device of the present invention maintains a true cylindrical shape ideal for rotating with no protruding buttons or clasps. The device can be placed at any point on the guide wire rather than only back loaded as are many current devices and fixates the wire in a central location firmly allowing proper center of rotation and adequate torque. The device can also wet and wipe the guide wire by with an absorbent foam in the housing that releases moisture when squeezed to reposition it along the wire.

In one embodiment, the present invention is a guide-wire or catheter torque device comprising: a torsion spring comprising a spring and one or more handles; one or more arms connected to the torsion spring and one or more elastomeric guide-wire or catheter grippers, wherein compression of the one or more handles reduced the compression between the one or more elastomeric grippers and a guide-wire or catheter; and an elongated, hollow housing comprising a first end and a second end, wherein the housing surrounds the spring tension metal clip comprising two or more opposing flexible pads positioned adjacent the spring tension handles that allow compression of the spring tension handles, the housing comprising a guide-wire or catheter guiding groove that extends from the first to the second end of the housing, wherein the one or more elastomeric grippers are aligned with and are positioned about the groove, wherein the one or more elastomeric grippers contact and grip a guide wire or catheter in the groove when the one or more spring tension handles are not engaged, but release variably as the user presses the flexible pads on the housing. In one aspect, the elastomeric grippers further comprise a backing that provides additional mechanical strength to the elastomeric wire-grippers. In another aspect, the housing comprises one or more protrusions at the first, the second, or both ends adjacent the guide-wire or catheter groove. In another aspect, the elastomeric grippers comprise one or more strips or bands of elastomeric material.

In yet another aspect, the elastomeric grippers are further defined as a compressible and resilient material comprising one or more of PVC, a natural or synthetic polymer, a natural or synthetic rubber, thermoplastic polyurethane, poly(ether-amide) block copolymer, thermoplastic rubber, styrene-butadiene copolymer, silicon rubber, synthetic rubber, styrene isoprene copolymer, styrene ethylene butylene copolymer, butyl rubber, nylon copolymer, spandex fibers comprising segmented polyurethane, ethylene-vinyl acetate copolymer or mixtures thereof. In one example, the elastomeric grippers comprise a silicone-modified polymer selected from the group consisting essentially of polyurethane, acrylic, vinyl, alkyl, ester and EPDM, and combinations thereof. In one aspect, torsion spring has a compression of between 0.1 and 8 ft/lbs of pressure to open the elastomeric grippers. In another aspect, the elastomeric grippers further comprise a backing that provides additional mechanical strength to the one or more elastomeric wire-gripper and an elastomeric material is coated on the backing to form the elastomeric wire-gripper. One example of materials for the housing comprises a natural rubber, polyvinyl, polyurethane, synthetic rubber, latex, polyacrylates, polybutadiene, urethane resins, styrene-butadiene copolymers, acrylonitrile butadiene rubber, nylon, polyester, neoprene, metal, and combinations thereof. In another aspect, the housing further comprises a wettable material is selected from foam, gauze, polyethylene oxide, polyethylene glycol, polyvinyl alcohol, polyvinyl pyrolidone, polyvinyl pyridine, or mixtures thereof between the housing and the spring-tensioned metal clip. Non-limiting examples of guide-wire or catheter include metal wire, fiber optic, steerable guide wires, endovascular guide wires, surgical rods, catheters, guide-wire delivered stents, helical wires, surgical springs, surgical coils, cannulas, surgical ports, wire sutures, electrical wires, ultrasound probes and needles.

Another embodiment of the present invention is a self-tensioning apparatus for controlling the advancement and rotation of a surgical guideware comprising: a torsion spring comprising one or more torsion spring handles; one or more arms connected to the torsion spring and one or more elastomeric guideware grippers, wherein compression of the one or more torsion spring handles reduced the compression between the one or more elastomeric grippers and the guideware; and an elongated, hollow housing comprising a first end and a second end, wherein the housing surrounds the spring tension metal clip comprising two or more opposing flexible pads positioned adjacent the spring tension handles that allow compression of the spring tension handles, the housing comprising a guideware guiding groove that extends from the first to the second end of the housing, wherein the one or more elastomeric guideware-grippers are aligned with and are positioned about the groove, wherein the one or more elastomeric grippers contact and grip the guideware in the groove when the one or more spring tension handles are not engaged, but release variably as the user compresses the flexible pads on the housing. In one aspect, the elastomeric grippers further comprise a backing that provides additional mechanical strength to the elastomeric grippers. In another aspect, the housing comprises one or more protrusions at the first, the second, or both ends adjacent the guideware groove. The elastomeric grippers may comprise one or more strips or bands of elastomeric material. In one aspect, the elastomeric grippers are further defined as a compressible and resilient material comprising one or more of PVC, a natural or synthetic polymer, a natural or synthetic rubber, thermoplastic polyurethane, poly(ether-amide) block copolymer, thermoplastic rubber, styrene-butadiene copolymer, silicon rubber, synthetic rubber, styrene isoprene copolymer, styrene ethylene butylene copolymer, butyl rubber, nylon copolymer, spandex fibers comprising segmented polyurethane, ethylene-vinyl acetate copolymer or mixtures thereof. Other examples of elastomeric grippers include a silicone-modified polymer selected from the group consisting essentially of polyurethane, acrylic, vinyl, alkyl, ester and EPDM, and combinations thereof. Generally, spring compression may be of between 0.1 and 8 ft/lbs of pressure to open the elastomeric grippers.

In one aspect of the present invention, the elastomeric grippers further comprise a backing that provides additional mechanical strength to the one or more elastomeric wire-gripper and an elastomeric material is coated on the backing to form the elastomeric wire-gripper. In another aspect, the housing comprises a natural rubber, polyvinyl, polyurethane, synthetic rubber, latex, polyacrylates, polybutadiene, urethane resins, styrene-butadiene copolymers, acrylonitrile butadiene rubber, nylon, polyester, neoprene, metal, and combinations thereof. The housing may further comprise a wettable material is selected from foam, gauze, polyethylene oxide, polyethylene glycol, polyvinyl alcohol, polyvinyl pyrolidone, polyvinyl pyridine, or mixtures thereof between the housing and the spring-tensioned metal clip. Non-limiting examples of guideware for control using the present invention include metal wires, fiber optic, steerable guide wires, endovascular guide wires, surgical rods, catheters, guide-wire delivered stents, helical wires, surgical springs, surgical coils, cannulas, surgical ports, wire sutures, electrical wires, ultrasound probes and needles.

In another embodiment, the present invention is a kit comprising a self-tensioning apparatus for controlling the advancement and rotation of a surgical guideware comprising: a torsion spring comprising one or more torsion spring handles; one or more arms connected to the torsion spring and one or more elastomeric guideware grippers, wherein compression of the one or more torsion spring handles reduced the compression between the one or more elastomeric grippers and the guideware; and an elongated, hollow housing comprising a first end and a second end, wherein the housing surrounds the spring tension metal clip comprising two or more opposing flexible pads positioned adjacent the spring tension handles that allow compression of the spring tension handles, the housing comprising a guideware guiding groove that extends from the first to the second end of the housing, wherein the one or more elastomeric guideware-grippers are aligned with and are positioned about the groove, wherein the one or more elastomeric grippers contact and grip the guideware in the groove when the one or more spring tension handles are not engaged, but release variably as the user compresses the flexible pads on the housing in a sterile packaging.

The present invention also includes a method of making a self-tensioning apparatus for controlling the advancement and rotation of a surgical guideware comprising: connecting to a torsion spring comprising one or more torsion spring handles; one or more arms and one or more elastomeric guideware grippers, wherein compression of the one or more torsion spring handles reduced the compression between the one or more elastomeric grippers and the guideware to form a torsion spring assembly. Next, placing the torsion spring assembly in an elongated, hollow housing comprising a first end and a second end, wherein the housing surrounds the spring tension metal clip comprising two or more opposing flexible pads positioned adjacent the spring tension handles that allow compression of the spring tension handles, the housing comprising a guideware guiding groove that extends from the first to the second end of the housing, wherein the one or more elastomeric guideware-grippers are aligned with and are positioned about the groove, wherein the one or more elastomeric grippers contact and grip the guideware in the groove when the one or more spring tension handles are not engaged, but release variably as the user compresses the flexible pads on the housing.

### BRIEF DESCRIPTION OF THE DRAWINGS

For a more complete understanding of the features and advantages of the present invention, reference is now made to the detailed description of the invention along with the accompanying figures and in which:

FIG. 1 is a top view of a guide clip of the present invention, illustrating the inside elements and outer cover;

FIG. 2 is a side view of the guide clip;

FIG. 3 is a bottom view of the device;

FIG. 4 is a front view of the guide clip;

FIG. 5 is an end view of the guide clip attached to a guide wire;

FIG. 6 is an end view of the guide clip;

FIG. 7 is an end-on view of the inner spring clip mechanism in which the elastomeric materials has a groove;

FIG. 7 is an end-on view of the inner spring clip mechanism having four sections; and

FIG. 9 is a cut-out, side view of another embodiment of the inner spring clip mechanism in a cut-out housing.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention relates to a medical device called a Glide Clip that may be used in the surgical theater to control the position and rotation of guideware. The Guide Clip is a guide wire torque device that is different from existing torque devices due to its ability to be placed onto any point along the guide wire rather than only over the back end of the guide wire, to be easily moved with one hand while maintaining cylindrical shape ideal for rotating, to wet and wipe a hydrophilic guide wire, and to be available in different sizes to better fit different guide wires. These features are made possible due to the spring tensioning mechanism. The device can be “clipped” on the wire rather than back loaded onto it. The device is designed to be produced in a variety of sizes to fit different guide wires and operator preferences. The housing has, internally, a torsion spring (e.g., a spring-loaded clip) that compresses one or more elastomeric guideware grippers that compress and mechanically restrain any endovascular guideware (e.g., guide wires, catheters and the like) for the purpose of rotating and advancing the guidewire. The main component is a torsion loaded spring assembly that tensions “jaws” that engage the guideware when released, and release the guideware when a housing in which the torsion loaded spring assembly is squeezed allowing repositioning of the device along the guideware. The torsion loaded spring assembly is housed in and attached to a plastic or metal tubular case that is designed to be produced in a variety of sizes. The elastomeric grippers apply force to the guide wire through the self tension of the spring which is released by squeezing the rear flexible area of the housing, which allows removal or movement along the guide wire as it is advanced through the blood vessel.

In one embodiment, the torsion spring loaded assemble has only one elastomeric gripper that compresses against a guideware groove in the housing, thereby using the torsion spring to restrain the guideware. The device of the present invention can be formed using one or more elastomeric grippers, depending on the size and physical-mechanical shape and strength of the guideware. Generally, the tension on the guideware will be such that it permits a firm grip on the guideware when the user removes the pressure on one or more external pads on the housing that are connected to the handles of the torsion spring. However, the tension must not be so large that it deforms the guideware during use, as the bending, kinking or collapse of the guideware is generally contraindicated for the integrity of the guideware.

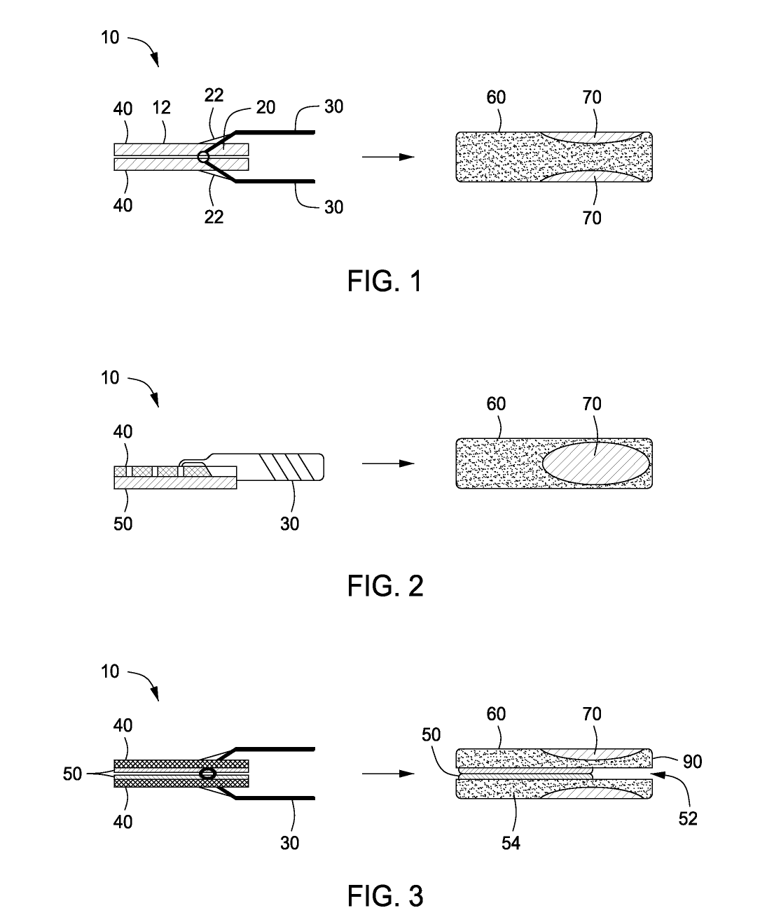

The torsion spring may be made from a wire size ranging from 0.008 to 0.500 inches, and may be left handed or right handed. Examples of metal for making the torsion spring include: surgical steel, Chromalloy, plastic, music wire, hard drawn wire, Phosphor Bronze, Beryllium Copper, Stainless Steel: 302, 304, 316, 17-7PH, 455 grades, MP35N, Nitinol—memory & superelastic, Nichrome, Magnet wire, MRI compatible alloys, Tungsten, Molybdenum, Titanium, Gold, Platinum Iridium alloy, Nimic, Hastelloy, Waspalloy, Inconel 600, X750, Ni-Span, Eligiloy, other high temperature alloys, which may also be coated. The torsion spring may be obtained from, e.g., Gifford Spring Company, Garland, Tex. Different wires may be selected depending on the compression required for gripping the guide-wire or catheter. In particular with catheters, the strength of the compression, the shape of the grippers and the contact surface between the grippers and the catheter may be varied or selected depending on the compression strength of the catheter, that is, the pressure applied by the gripper(s) will be selected to be less than the pressure that would collapse the lumen of the catheter. Examples of material that may be used to make the torsion spring, e.g., the spring depicted in FIG. 1, may be: music wire a high carbon steel for high stress applications; harddrawn MB which is a carbon steel for low stresses and low cost; oil tempered wire which is a carbon steel for wire forms and torsion springs; stainless steel which is types 302 and 316 for high stresses in larger wire sizes; chrome vanadium which is an alloy wire for high stresses in larger wire sizes; phosphor bronze which is for electrical applications or even; brass which is for applications requiring water resistance. The metal will generally be coated to be biocompatible, e.g., Zinc plate, black oxide, shot peen, passivate, as well as any other required, in particular, biocompatible coatings. Other examples of coating may include, e.g., bioactive agents, non-allergenic agents, anti-clotting agents, anti-bacterial agents.

The elastomeric material used in the grippers to grip the guideware is selected from any material that is compressible and resilient. Non-limiting examples of elastomeric materials include, a natural or synthetic polymer, a natural or synthetic rubber, thermoplastic polyurethane, poly(ether-amide) block copolymer, thermoplastic rubber, styrene-butadiene copolymer, silicon rubber, synthetic rubber, styrene isoprene copolymer, styrene ethylene butylene copolymer, butyl rubber, nylon copolymer, spandex fibers comprising segmented polyurethane, ethylene-vinyl acetate copolymer, PVC or mixtures thereof.

The housing may be made from any biocompatible material, including but not limited to: natural rubber, polyvinyl, polyurethane, synthetic rubber, latex, polyacrylates, polybutadiene, urethane resins, styrene-butadiene copolymers, acrylonitrile butadiene rubber, nylon, polyester, neoprene, metal, and combinations thereof. When the housing is substantially hollow (as opposed to only providing space for the internal compression mechanism, the housing may include therein a wettable material is selected from foam, gauze, polyethylene oxide, polyethylene glycol, polyvinyl alcohol, polyvinyl pyrolidone, polyvinyl pyridine, or mixtures thereof between the housing and the spring-tensioned metal clip.

As used herein, the term “guideware” refers to a tool controlled by the present invention, e.g., inserting into a patient, withdrawing from a patient, rotating the guideware or combinations thereof. Example of guideware include, but are not limited to, guide wires, catheters, fiber optic, steerable guide wires, endovascular guide wires, surgical rods, catheters, guide-wire delivered stents, helical wires, surgical springs, surgical coils, cannulas, surgical ports, wire sutures, electrical wires, ultrasound probes and needles. Guideware may be plastic, metal, polymeric, hydrophobic, hydrophilic, biocompatible, biodegradable and combinations thereof, as necessary for surgical procedures requiring an at least partially guided surgical instrument that can be gripped and controlled with one hand as described and its use taught herein.

The torsion spring tensioned clip is composed of two metal arms attached to one or more torsion (or coiled) spring(s) that allows levered movement of the arms. One end of the arms comes in contact with force derived from the spring. Gripping jaws with textured rubber attach directly and firmly to the guide wire. The ends that come into contact are the point of attachment to the guide wire. The opposite ends of the arms release the force of attachment when squeezed together. This spring tensioned clip is attached to the outer covering or housing via a central core of material that is extruded through the center of the coil of the spring allowing just enough clearance on the inside of the housing or covering for movement of the arms. The back end of the arms that are squeezed together rest adjacent to flexible areas in the back of the housing which is also filled with absorbent foam material. The outer housing contains a slot or groove that allows passage of the guide wire into the center of the device.

While the present invention has been described in terms of specific embodiments, it is to be understood that the invention is not limited to these disclosed embodiments. This invention may be embodied in many different forms and should not be construed as limited to the embodiments set forth herein; rather, these embodiments are provided by way of illustration only and so that this disclosure will be thorough, complete and will fully convey the full scope of the invention to those skilled in the art. Indeed, many modifications and other embodiments of the invention will come to mind of those skilled in the art to which the invention pertains, and which are intended to be and are covered by this disclosure, the drawings and claims. It may be used on any guide wire for any endovascular or interventional procedure.

FIG. 1 (left side) is a top view of the internal portions of the device 10 of the present invention, with the outer cover (right) removed showing detail of the inner spring clip mechanism 12. The components of the inner spring clip mechanism 12 include a torsion spring 20, connected to handles 30, elastomeric grippers or material 40 connected to a tensioned spring 20 by arms 22. The housing 60 is depicted with two flexible pads or soft grips 70. The inner spring clip mechanism 12 is aligned inside the housing 60 so that the handles 30 align with the pads 70.

FIG. 2 is a side view of the device 10 with the housing 60 removed (left panel) showing the detail of the inner spring clip mechanism 12. The side view shows the elastomeric grippers 40, in this embodiment having a solid backing or support 50, with multiple bands or striations for the elastomeric grippers 40. The right panel shows the positioning of the pads 70 with regard to the surface area of the handles 30, the alignment is best seen in this view.

FIG. 3 is a bottom view of the device 10 with housing 60 removed (left panel) showing the alignment of the inner spring clip mechanism 12, the elastomeric grippers 40, and the guideware groove 52 in the housing 60. The groove 52 is aligned with the elastomeric grippers 40, the handles 30 and the pads 70 wherein the guideware fits in the guideware groove 52, without the torsion spring 20 or the arms 22, interfereing with the contact between the elastomeric grippers 40 and a guideware positioned in the guideware groove 52 between the elastomeric grippers 40. Also shown is the inside of the guideware groove 52 in the housing (60) (right panel), which can contain an absorbent material 54 that can carry moisture and wet and wipe the guideware.

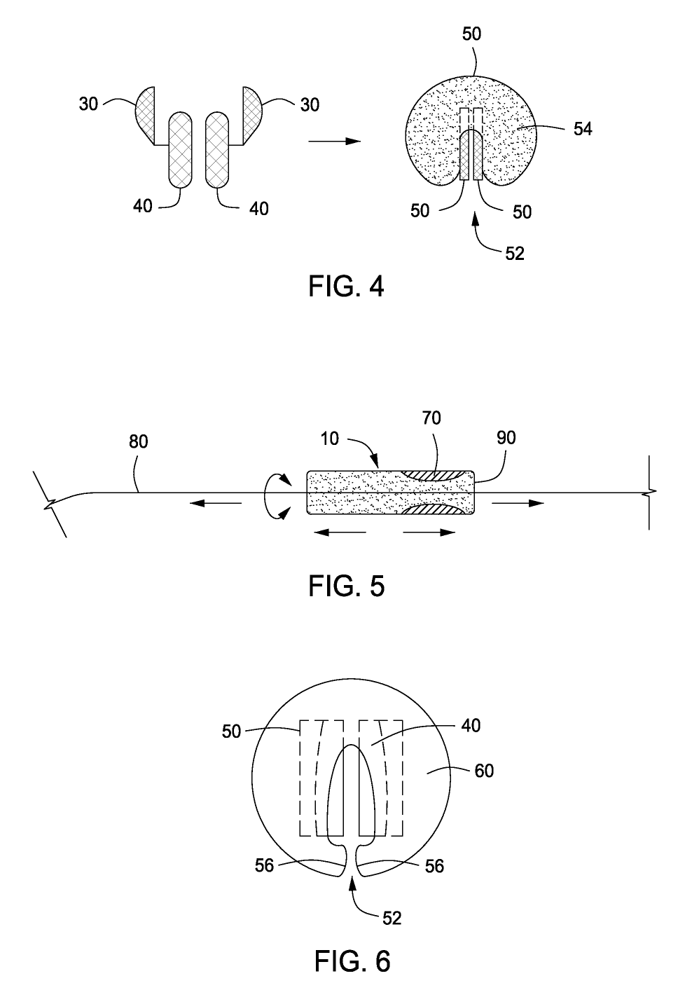

FIG. 4 is an end view of the device with the inner spring clip mechanism 12 (left panel) removed and from the housing 60 (right panel). This view demonstrates the elongated or cylindrical shape of the housing 60 and the guideware groove 52 and, if applicable, the absorbent material 54. The elastomeric grippers 40 are shown partially open in relation to the handles 30.

FIG. 5 is a schematic drawing of the device 10 as it would attach to, in this view, a guide wire 80. The operator would squeeze, compress or depress the pads 70 on the surface of the housing 60 to cause the release of the wire 80 by the elastomeric grippers in the housing 60. The device 10 can them be moved anywhere along guidewire 80. When the pads 70 are not compressed, the elastomeric grippers 40 once again grip the guide wire 80 via the compression cause between the elastomeric grippers 40 caused by the torsion spring 20 via arms 22. Once the elastomeric grippers 40 have compressed onto the guide wire 80, then the user can push, pull and or rotate the guide wire 80 by exerting the proper motion, with only one hand, on the housing. If the operator wants to have partial pressure on the guide wire 80, then the operator can partially depress the pads 70 to release some of the pressure exerted to restrain guide wire 80, while concurrently performing a push, pull or rotation.

FIG. 6 is another end view of the device 10 of the present invention which shows the addition of one or more protrusions 56 at the tip of the ends of the housing 60. The protrusions can be used to partially restrain the guide wire in the guideware groove 52 so that the device 10 can be moved up and down the guide wire without releasing the guidewire. The guidewire can be inserted and removed with a slight addition of pressure when inserting or releasing. The protrusions 56 can be of any shape, for example, nubs, teeth, sawteeth or cones. The protrusions 56 can be from the same or a separate material as the housing 60, and can be molded into the housing or added after the housing 60 is made.

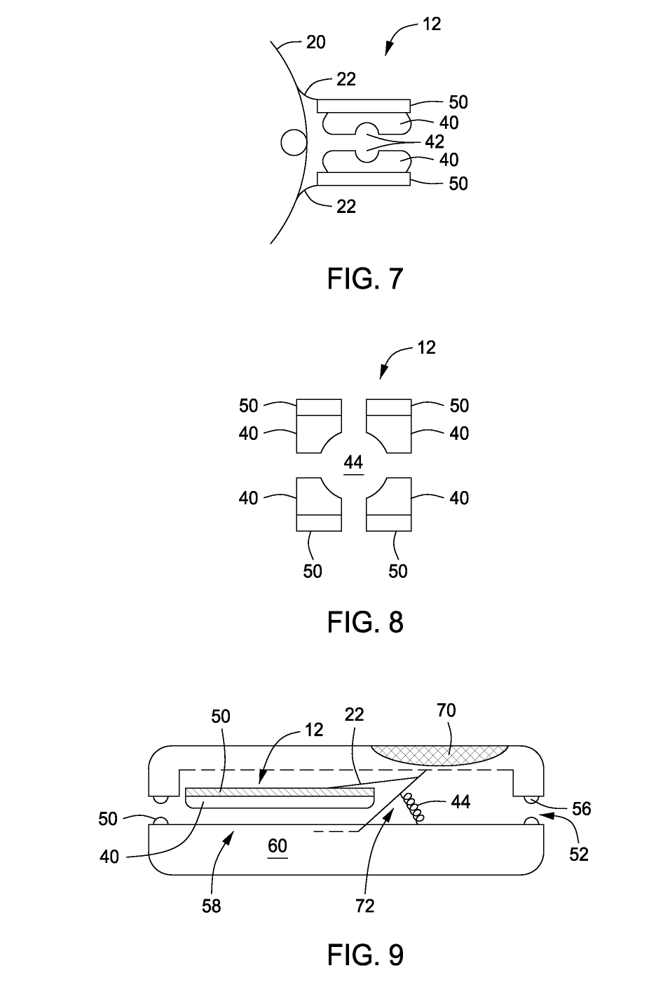

FIG. 7 is an end view of the device with the inner spring clip mechanism 12, also showing the torsion spring 20, connected to arms 22, which in this embodiment connect to backing 50. The elastomeric material 40, is depicted in this embodiment having a groove 42 into which a guideware, such as a catheter having a lumen (not depicted), is inserted and provides increased surface area surrounding the guideware to provide a better grip.

FIG. 8 is an end view of the device with the inner spring clip mechanism 12, in this embodiment; four separate backing 50/elastomeric material 40 sections are shown which can provide compression from multiple directions. In this embodiment having an opening 44 is formed by the four separate backing 50/elastomeric material 40 sections. From this figure, the skilled artisan will appreciate that the backing 50/elastomeric material 40 sections can be any of a number of integers (1, 2, 3, 4, 5, 6, 7, 8, 9, 10, etc.) and can also be of varying sizes (not depicted). For example, a pair of backing 50/elastomeric material 40 sections could have, on one side a protrusion on the surface of the elastomeric material that contacts the guideware, and on the opposite side a matching groove.

FIG. 9 is a cut-out, side view of another embodiment of the inner spring clip mechanism 12 in a cut-out housing 60, in which the elastomeric material 40, on backing 50 is depicted and in which a single elastomeric material 40 gripper compresses against a side 58 of the groove 52 and has another compression spring mechanism 12, in this case having a single pivot arm 72 embedded in the housing 60 and having a extension spring 44. Also shown are pairs of protrusions 56 at the first and second ends of the housing 60 at groove 52.

In operation, a guide wire torque device that is different from existing torque devices due to its ability to be placed over the guide wire rather than only over the back end of the guide wire and also due to its ability to be moved with one hand while maintaining a cylindrical shape ideal for rotating. It also wets and wipes or cleans the wire each time it is advanced over the wire and it is designed to be produced in different sizes to fit different wires or operator preference.

A torque device that is a spring tensioned clip that is housed in a plastic covering with opposing flexible soft gripping areas that allow squeezing of the clip releasing tension of the spring thereby allowing attachment and removal from a guiding wire.

It works by opening the spring loaded jaws that allow it to be placed on the guide wire and is self tensioning.

It can be released completely or partially by squeezing it thereby opening the jaws allowing removal or repositioning along the guide wire.

The inside of the housing is filled with absorbent foam material that when squeezed to reposition releases moisture to wet the wire.

Guide wires are integral to every interventional cardiology and radiology (endovascular) procedure. They allow passage of catheters for angiograms and angioplasties as well as many other procedures. Guide wires allow the navigation of the vessel being studied or treated and torque (rotation) is vital to navigation through vessels. This device aids in that navigation by allowing one handed movement of the torque device as the vessel is navigated.

In operation, the present invention functions as follows, when the surgeon compresses the opposing flexible pads on the housing, the torsion spring is compressed and the elastomeric guide-wire or catheter grippers are separated thereby reducing their grip on the medical endovascular guide wire, catheter or other surgical instrument. The housing can be solid or hollow and cam be filled with an absorbent foam, gauze or other hydrophilic or hydrophobic material that when wet will both wet and wipe a guide wire, generally, hydrophilic surgical instrument that is inserted through a small opening or port in the patient.

The device allows the user to have single hand attachment for interventional radiology/cardiology (endovascular) guide wires (or other instruments) that permits torqueing (rotating) and advancing the guide wire, without having to remove their control over, e.g., the entire guide wire as the torque device is moved along the guide wire and to have better control over the compression of the elastomeric guide-wire or catheter grippers on the surgical tool. Therefore, the device can be placed on and/or moved along the guide wire (or even removed) with one hand rather than all current devices which require releasing the guide wire in order to have both hands free to move, place and torque down existing devices. The device of the present invention maintains a true cylindrical shape ideal for rotating with no protruding buttons or clasps, thereby, not damaging the wire and/or compression the lumen of a catheter or other surgical device. The device can be placed rapidly, at any point on the guide wire or catheter, with a single hand. The device of the present invention can also wet and wipe the guide wire or catheter during actual use with an absorbent foam, gauze or material in the housing that releases moisture when squeezed to reposition it along the wire.

When performing surgery that includes the use of two or more guide wires, catheters or combinations thereof, the present invention allows the user to have full control of two or more guide wire and/or catheters single handedly, thereby reducing the number of users in the surgical field (thereby reducing costs and the possibility of exposure to a new source of possible infection), has improved “hand feel” and control, as well as providing the user with greatly improved control over the guide wire or catheter during surgery.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A guide-wire or catheter torque device comprising:
a torsion spring comprising a spring and one or more handles;
one or more arms connected to the torsion spring and one or more elastomeric guide-wire or catheter grippers, wherein compression of the one or more handles reduced the compression between the one or more elastomeric grippers and a guide-wire or catheter; and
an elongated, hollow housing comprising a first end and a second end, wherein the housing surrounds the spring tension metal clip comprising two or more opposing flexible pads positioned adjacent the spring tension handles that allow compression of the spring tension handles, the housing comprising a guide-wire or catheter guiding groove that extends from the first to the second end of the housing, wherein the one or more elastomeric grippers are aligned with and are positioned about the groove, wherein the one or more elastomeric grippers contact and grip a guide wire or catheter in the groove when the one or more spring tension handles are not engaged, but release variably as the user presses the flexible pads on the housing.

2. The torque device of claim 1, wherein the elastomeric grippers further comprise a backing that provides additional mechanical strength to the elastomeric wire-grippers.

3. The torque device of claim 1, wherein the housing comprises one or more protrusions at the first, the second, or both ends adjacent the guide-wire or catheter groove.

4. The torque device of claim 1, wherein the elastomeric grippers comprise one or more strips or bands of elastomeric material.

5. The torque device of claim 1, wherein the elastomeric grippers are further defined as a compressible and resilient material comprising one or more of PVC, a natural or synthetic polymer, a natural or synthetic rubber, thermoplastic polyurethane, poly(ether-amide) block copolymer, thermoplastic rubber, styrene-butadiene copolymer, silicon rubber, synthetic rubber, styrene isoprene copolymer, styrene ethylene butylene copolymer, butyl rubber, nylon copolymer, spandex fibers comprising segmented polyurethane, ethylene-vinyl acetate copolymer or mixtures thereof.

6. The torque device of claim 1, wherein the elastomeric grippers comprise a silicone-modified polymer selected from the group consisting essentially of polyurethane, acrylic, vinyl, alkyl, ester and EPDM, and combinations thereof.

7. The torque device of claim 1, wherein the spring requires a compression of between 0.1 and 8 ft/lbs of pressure to open the elastomeric grippers.

8. The torque device of claim 1, wherein the elastomeric grippers further comprise a backing that provides additional mechanical strength to the one or more elastomeric wire-gripper and an elastomeric material is coated on the backing to form the elastomeric wire-gripper.

9. The torque device of claim 1, wherein the housing comprises a natural rubber, polyvinyl, polyurethane, synthetic rubber, latex, polyacrylates, polybutadiene, urethane resins, styrene-butadiene copolymers, acrylonitrile butadiene rubber, nylon, polyester, neoprene, metal, and combinations thereof.

10. The torque device of claim 1, wherein the housing further comprises a wettable material is selected from foam, gauze, polyethylene oxide, polyethylene glycol, polyvinyl alcohol, polyvinyl pyrolidone, polyvinyl pyridine, or mixtures thereof between the housing and the spring-tensioned metal clip.

11. The torque device of claim 1, wherein the guide-wire or catheter is selected from a fiber optic, steerable guide wires, endovascular guide wires, surgical rods, catheters, guide-wire delivered stents, helical wires, surgical springs, surgical coils, cannulas, surgical ports, wire sutures, electrical wires, ultrasound probes and needles.

12. A self-tensioning apparatus for controlling the advancement and rotation of a surgical guideware comprising:
a torsion spring comprising one or more torsion spring handles;
one or more arms connected to the torsion spring and one or more elastomeric guideware grippers, wherein compression of the one or more torsion spring handles reduced the compression between the one or more elastomeric grippers and the guideware; and
an elongated, hollow housing comprising a first end and a second end, wherein the housing surrounds the spring tension metal clip comprising two or more opposing flexible pads positioned adjacent the spring tension handles that allow compression of the spring tension handles, the housing comprising a guideware guiding groove that extends from the first to the second end of the housing, wherein the one or more elastomeric guideware-grippers are aligned with and are positioned about the groove, wherein the one or more elastomeric grippers contact and grip the guideware in the groove when the one or more spring tension handles are not engaged, but release variably as the user compresses the flexible pads on the housing.

13. The apparatus of claim 12, wherein the elastomeric grippers further comprise a backing that provides additional mechanical strength to the elastomeric grippers.

14. The apparatus of claim 12, wherein the housing comprises one or more protrusions at the first, the second, or both ends adjacent the guideware groove.

15. The apparatus of claim 12, wherein the elastomeric grippers comprise one or more strips or bands of elastomeric material.

16. The apparatus of claim 12, wherein the elastomeric grippers are further defined as a compressible and resilient material comprising one or more of PVC, a natural or synthetic polymer, a natural or synthetic rubber, thermoplastic polyurethane, poly(ether-amide) block copolymer, thermoplastic rubber, styrene-butadiene copolymer, silicon rubber, synthetic rubber, styrene isoprene copolymer, styrene ethylene butylene copolymer, butyl rubber, nylon copolymer, spandex fibers comprising segmented polyurethane, ethylene-vinyl acetate copolymer or mixtures thereof.

17. The apparatus of claim 12, wherein the elastomeric grippers comprise a silicone-modified polymer selected from the group consisting essentially of polyurethane, acrylic, vinyl, alkyl, ester and EPDM, and combinations thereof.

18. The apparatus of claim 12, wherein the spring requires a compression of between 0.1 and 8 ft/lbs of pressure to open the elastomeric grippers.

19. The apparatus of claim 12, wherein the elastomeric grippers further comprise a backing that provides additional mechanical strength to the one or more elastomeric wire-gripper and an elastomeric material is coated on the backing to form the elastomeric wire-gripper.

20. The apparatus of claim 12, wherein the housing comprises a natural rubber, polyvinyl, polyurethane, synthetic rubber, latex, polyacrylates, polybutadiene, urethane resins, styrene-butadiene copolymers, acrylonitrile butadiene rubber, nylon, polyester, neoprene, metal, and combinations thereof.

21. The apparatus of claim 12, wherein the housing further comprises a wettable material is selected from foam, gauze, polyethylene oxide, polyethylene glycol, polyvinyl alcohol, polyvinyl pyrolidone, polyvinyl pyridine, or mixtures thereof between the housing and the spring-tensioned metal clip.

22. The apparatus of claim 12, wherein the guideware is selected from a fiber optic, steerable guide wires, endovascular guide wires, surgical rods, catheters, guide-wire delivered stents, helical wires, surgical springs, surgical coils, cannulas, surgical ports, wire sutures, electrical wires, ultrasound probes and needles.

23. A kit comprising a self-tensioning apparatus for controlling the advancement and rotation of a surgical guideware comprising:
a torsion spring comprising one or more torsion spring handles;
one or more arms connected to the torsion spring and one or more elastomeric guideware grippers, wherein compression of the one or more torsion spring handles reduced the compression between the one or more elastomeric grippers and the guideware; and
an elongated, hollow housing comprising a first end and a second end, wherein the housing surrounds the spring tension metal clip comprising two or more opposing flexible pads positioned adjacent the spring tension handles that allow compression of the spring tension handles, the housing comprising a guideware guiding groove that extends from the first to the second end of the housing, wherein the one or more elastomeric guideware-grippers are aligned with and are positioned about the groove, wherein the one or more elastomeric grippers contact and grip the guideware in the groove when the one or more spring tension handles are not engaged, but release variably as the user compresses the flexible pads on the housing in a sterile packaging.
